(19) United States
(12) Patent Application Publication
(10) Pub. No.: US 2005/0226932 A1
(43) Pub. Date: Oct. 13, 2005

(54) Pharmaceutical formulations for itraconazole

(75) Inventors: Hye-Jeong Yoon, Daejeon (KR); Min Hyo Seo, Daejeon (KR); Jeong-Kyung Kim, Daejeon (KR)

Correspondence Address: THORPE NORTH & WESTERN, LLP., 8180 SOUTH 700 EAST, SUITE 200, P.O. BOX 1219, SANDY, UT, 84070, US

(73) Assignee: SAMYANG CORPORATION

(21) Appl. No.: 11/049,266

(22) Filed: Feb. 1, 2005

Related U.S. Application Data
(63) Continuation-in-part of application No. 10/492,091, filed on Apr. 9, 2004.
(63) Continuation-in-part of application No. 10/493,043, filed on Apr. 15, 2004.
(63) Continuation-in-part of application No. 10/962,204, filed on Oct. 7, 2004.

(51) Int. Cl. A61K 31/496; A61K 9/14; A61K 9/50

(52) U.S. Cl. 424/486; 514/254.07

(57) ABSTRACT

The present invention relates to a pharmaceutical composition containing as an active ingredient, itraconazole. More particularly, the present invention relates to the pharmaceutical composition comprising itraconazole, a polylactic acid derivative, and an amphiphilic block copolymer. The polylactic acid derivative may be combined with a metal ion at the carboxylic acid terminal, and the amphiphilic block copolymer forms micelles or nano-particles in an aqueous medium.

### CROSS-REFERENCE TO RELATED APPLICATIONS AND CLAIM OF PRIORITY

This application is a continuation in-part of U.S. patent application Ser. Nos. 10/492,091 filed on Apr. 9, 2004, 10/493,043 filed on Apr. 15, 2004 and 10/962,204 filed on Oct. 7, 2004.

### BACKGROUND

Itraconazole or (±)-Cis-4-[4-[4-[4-[[2-(2,4-dichlorophenyl)-2-(1H-1,2,4-triazol-1-ylmethyl)-1,3-dioxolan-4-yl]methoxy]phenyl]-1-piperazinyl]phenyl]-2,4-dihydro-2-(1-methylpropyl)-3H-1,2,4-triazol-3-one is one of many broad spectrum antifungal compounds, and is water insoluble. Other water insoluble azole compounds include oxiconazole, bifonazole, isoconazole, isoconazole nitrate, terconazole, clotrimazole, econazole nitrate, ketoconazole, miconazole, miconazole nitrate, sertaconazole nitrate, itraconazole and saperconazole. The molecular formula of itraconazole is C₃₅H₃₀Cl₂N₈O₄ and its molecular weight is 705.64. It is white or pale yellow powder and practically insoluble in water (less than 1 μg/Ml), very slightly soluble in alcohol (300 μg/Ml), and freely soluble in methylene chloride (239 mg/Ml). It is a weakly basic compound having a pKa value of 3.7 and is almost completely ionized in strongly acidic conditions, such as gastric juice. It is known in the pharmacological field to provide a broad spectrum of antifungal activity in oral formulations, in parenteral preparations, and in topical preparations. The effective blood concentration of itraconazole is 0.25-0.5 mcg/mL (The Annals of Pharmacotherapy. 2001, June, Vol 35).

The development of efficacious pharmaceutical formulations of azoles such as itraconazole is hampered considerably by the fact that azoles are only very sparingly soluble in water. Since itraconazole has an extremely low solubility, which is dependent on pH, it is difficult to prepare formulations of the drug.

Since such insoluble drugs generally dissolve slowly from solid dosage forms, dissolution thereof is the rate-limiting step in absorption of the drug. The dissolution rate directly affects the onset of action, intensity of action, and duration of action. Therefore, demand for increasing the dissolution rate of such insoluble compounds is increasing.

The solubility and dissolution rate of such poorly soluble compounds can be increased by means of a reduction in particle size, polymorphism, amorphousness, power crushing, solid dispersing, inclusion complexing, solvated compounding, protein binding, interaction with additives and the like. However, even with various pharmaceutical methods, it is still not easy to enhance the dissolution rate of a particular drug when considering its specific physicochemical properties. Especially, in enhancing the dissolution rate of poorly water-soluble compounds, many pharmaceutical factors including easiness of the formulation process should be considered. PCT International Publication No. WO85/002767 discloses a method for improving the solubility of itraconzaole in water by forming inclusion compounds with cyclodextrin or its derivatives. PCT International Publication No. WO93/015719 discloses a method for liposomal preparation for external use containing itraconazole and phospholipids by a solvent system. WO96/039835 also discloses a method for adding itraconazole into a volatile organic solvent having a low molecular weight, such as acetic acid or formic acid.

However, these methods have problems such as insufficient increases in solubility, inappropriateness for oral administration, difficulty with preparation. As a result, they have still not gained much value as products.

On the other hand, PCT International Publication No. WO94/005263 discloses an oral bead-typed preparation with improved bioavailability and enhanced solubility, in which a hydrophilic polymer such as hydroxypropyl methylcellulose and a drug are coated with many small sugar spheres having a 25-30 mesh core. Janssen Pharmaceutica N. V. has developed the above and commercialized it as Sporanox® Capsule. However, it has some drawbacks in that the manufacturing process is complicated since Sporanox® capsules require a seal coating over the drug coating layer to prevent sticking of the beads, which is troublesome and would result in the undesirable effect of a concomitant decrease in the dissolution rate and bioavailability. In addition, the dissolution of the drug in the GI tract is significantly affected by food take. PCT International Publication No. WO97/044014 further discloses a method for preparing solid dispersions of itraconazole and a water-soluble polymer by a melt-extrusion method at a temperature of 245-265° C. and subsequently milling said melt-extruded mixture.

The above solid dispersion is characterized by an increased dissolution rate of drug and lowered food effect. But the manufacturing process at the higher temperature may affect the stability of the drug and there are difficulties in handling extruded products.

To solve the above mentioned problems, demands for the formulations containing itraconazole having both high stability and bioavailability by increasing the solubility of itraconazole and an easy preparation method thereof are rapidly increasing.

Korean Patent Publication No. 1999-51527 discloses technologies wherein the particle size of itraconazole is reduced and its crystallinity is changed by making a eutectic mixture and milling the itraconazole and water-soluble saccharides to increase the solubility and dissolution rate.

Korean Patent Publication No. 1999-62448 discloses a method of production and composition of an oral preparation of itraconazole with improved bioavailability by forming solid dispersions which is prepared by the following steps: i) dissolving 1 weight part of itraconazole and 0.5-5.0 weight parts of a hydrophilic polymer with solvent, ii) spray-drying said mixture, and iii) preparing the solid dispersions for oral preparation. These solid dispersions have amorphous round type particles with 1-5 μm of particle size distribution and are in contact closely with a hydrophilic carrier. Thus, they have increased solubility and initial dissolution rates.

Korean Patent Publication No. 1999-1564 discloses technologies designed to increase solubility and dissolution rate by reducing particle size and changing the crystallinity of poorly water-soluble itraconazole by using spray-drying. The particle diameter of itraconazole is in the range of 0.5-10 μm and the average particle diameter is about 3.7 μm which is reduced by a factor of 7 compared to that of itraconazole as the raw material. As a result, the solubility is increased by a factor of 62. However, it is disadvantageous in that the solubility is dependent on the spraying rate. If the spraying rate is too slow, it results in too much loss of drug due to fast evaporation of the solvent. If the spraying rate is too fast, on the other hand, an increase in particle size due to agglomeration of particles before the evaporation of the solvent results. Thus, since the manufacturing conditions highly depend on the particle size distribution, the particle size distribution and dissolution rate may not be uniform per each manufacture.

### SUMMARY

It has been recognized that it would be advantageous to develop a formulation containing itraconazole having both high stability and bioavailability by increasing the solubility of itraconazole and an easy preparation method thereof.

The present invention provides a pharmaceutical composition containing as an active ingredient, itraconazole. More particularly, the present invention relates to the pharmaceutical composition containing itraconazole as the active ingredient. The composition comprises itraconazole and a polylactic acid derivative having at least one terminal carboxyl group. One embodiment of the present invention comprises itraconazole, a polylactic acid derivative having at least one terminal carboxyl group, and an amphiphilic block copolymer. Another embodiment of the present invention comprises itraconazole, a polylactic acid derivative having at least one terminal carboxyl group wherein said carboxyl group is fixed with di-or tri-valent metal ion, and an amphiphilic block copolymer to form polymeric micelles or nano-particles in an aqueous medium.

The present invention provides a pharmaceutical composition having improved solubility and stability over those of conventional itraconazole preparations. The present invention also provides a pharmaceutical composition containing itraconazole as the active ingredient, wherein the composition effectively increases the solubility and stability of itraconazole in body fluids or aqueous solutions.

### BRIEF DESCRIPTION OF THE DRAWINGS

Additional features and advantages of the invention will be apparent from the detailed description which follows, taken in conjunction with the accompanying drawings, which together illustrate, by way of example, features of the invention.

The aforementioned aspects and other features of the present invention will be explained in the following description, taken in conjunction with the accompanying drawings, wherein:

FIG. 1 is a schematic diagram of a micelle of itraconazole entrapped in the hydrophobic core of a core-shell-typed carrier comprising an amphiphlic block copolymer and a polylactic acid derivative having the terminal carboxyl group positioned between the amphiphilic block copolymers in an aqueous environment.

FIG. 2 is a schematic diagram of a Ca⁺⁺-fixed polymeric micelle containing itraconazole trapped in the hydrophobic core of the micelle.

FIG. 3 is a graph representing changes in the itraconazole concentration in blood over time when the nano-particle composition prepared by Example 9 and a solubilized composition using cyclodextrin prepared by Comparative Example 2 of the present invention are intravenously administered; and

FIG. 4 is a graph representing changes in the concentration of itraconazole concentration in blood over various times when the micelle composition prepared by Example 2 and a solubilized composition using cyclodextrin prepared by Comparative Example 2 of the present invention are orally administered.

Reference will now be made to the exemplary embodiments illustrated, and specific language will be used herein to describe the same. It will nevertheless be understood that no limitation of the scope of the invention is thereby intended.

### DETAILED DESCRIPTION OF EXAMPLE EMBODIMENT(S)

Before the present polymeric compositions and methods of using and making thereof are disclosed and described, it should be understood that this invention is not limited to the particular configurations, process steps, and materials disclosed herein, and such configurations, process steps, and materials may be varied. It should be also understood that the terminology employed herein is used for the purpose of describing particular embodiments only and is not intended to limit the scope of the present invention which will be limited only by the appended claims and equivalents thereof.

It should be noted that, in this specification and the appended claims, the singular forms, “a,” “an,” or “the”, includes plural referents unless the context clearly dictates otherwise. Thus, for example, the reference to a polymer containing “a terminal group” includes reference to two or more such groups, and reference to “a hydrophobic drug” includes reference to two or more such drugs. Furthermore, reference to an amphiphilic block copolymer includes mixtures of block copolymers provided that the compositions of each A and B block, the respective ratios of each block, and weight or number average molecular weight of each block and/or the overall block polymeric composition fall within the limitations defined herein.

In describing and claiming the present invention, the following terminology will be used in accordance with the definitions set out below.

As used herein, the term “biodegradable” or “biodegradation” is defined as the conversion of materials into less complex intermediates or end products by solubilization hydrolysis, or by the action of biologically formed entities which can be enzymes or other products of the organism.

As used herein, the term “biocompatible” means materials or the intermediates or end products of materials formed by solubilization hydrolysis, or by the action of biologically formed entities which can be enzymes or other products of the organism and which cause no adverse effect on the body.

As used herein, “administering” and similar terms mean delivering the composition to an individual being treated such that the composition is capable of being circulated systemically. Preferably, the compositions of the present invention are administered by the subcutaneous, intramuscular, transdermal, oral, transmucosal, intravenous, or intraperitoneal routes. Injectables for such use can be prepared in conventional forms, either as a liquid solution or suspension, or in a solid form that is suitable for preparation as a solution or suspension in liquid prior to injection, or as an emulsion. Suitable excipients that can be used for administration include, for example, water, saline, dextrose, glycerol, ethanol, and the like; and if desired, minor amounts of auxiliary substances such as wetting or emulsifying agents, buffers, and the like. For oral administration, they can be formulated into various forms such as solutions, tablets, capsules, etc.

Below, the exemplary embodiments are shown and specific language will be used herein to describe the same. It should nevertheless be understood that no limitation of the scope of the invention is thereby intended. Alterations and further modifications of the inventive features illustrated herein, and additional applications of the principles of the present invention as illustrated herein, for one skilled in the relevant art, in connection with this disclosure, should be considered within the scope of the present invention.

The pharmaceutical composition of the present invention comprises itraconazole and a polylactic acid derivative having at least one terminal carboxyl group. Preferrably, the pharmaceutical composition of the present invention comprises itraconazole, a polylactic acid derivative having at least one terminal carboxyl group and an amphiphilic block copolymer. More preferably, the pharmaceutical composition of the present invention comprises itraconazole, a polylactic acid derivative having at least one terminal carboxyl group wherein said carboxyl group is fixed with a di- or tri-valent metal ion, and an amphiphilic block copolymer. Thus, the present composition may increase the solubility of water insoluble itraconazole by forming polymeric micelles or nano-particles in an aqueous medium and allows entrapment of a great quantity therein. This pharmaceutical composition allows itraconazole to be present in fine particle forms in an aqueous medium, and may be used for intravenous injection due to its high stability and prolonged circulation time in the blood stream.

The present invention provides a pharmaceutical composition comprising 0.1-30.0 wt. % of itraconazole, 70.0-99.9 wt. % of a polylactic acid derivative having at least one terminal carboxyl group. The present invention further provides a pharmaceutical composition comprising 0.1-30.0 wt. % of itraconazole, 5.0-99.8 wt. % of a polylactic acid derivative having at least one terminal carboxyl group, and 0.1-94.9 wt. % of an amphiphilic block copolymer. The present invention further provides a pharmaceutical composition comprising 0.1-30.0 wt. % of itraconazole, 5.0-99.8 wt. % of a polylactic acid derivative having at least one terminal carboxyl group wherein said carboxyl group is fixed with a di- or tri-valent metal ion, and 0.1-94.9 wt. % of an amphiphilic block copolymer. The polylactic acid derivative which forms polymeric micelles in the present invention preferably has number-average molecular weight of 500-2,500 Daltons, more preferably 800-1,500 Daltons.

Such polylactic acid derivative has a carboxylic acid or an alkali metal salt thereof combined at the terminal of polylactic acid and is chosen from D,L-polylactic acid, D-polylactic acid, polymandelic acid, a copolymer of D,L-lactic acid and glycolic acid, a copolymer of D,L-lactic acid and mandelic acid, a copolymer of D,L-lactic acid and caprolactone, a copolymer of D,L-lactic acid and 1,4-dioxane-2-one, and acyl-D,L-lactic acid substituted with a C₈-C₁₄ acyl including decanoyl, lauroyl, palmitoyl. Such alkali metal salt is chosen from monovalent metal ions such as sodium, potassium, and lithium.

Since the polylactic acid derivative has at least one carboxyl group or alkali metal salt thereof, the carboxylic acid or alkali metal salt thereof functions as a hydrophilic group in an aqueous solution with a pH of 4 or more and enables the polylactic acid derivative to form polymeric micelles therein.

While a pharmaceutical composition containing itraconazole forming polymeric micelles in an aqueous medium can be prepared under specific condition such as a pH of 4 or more when the polylactic acid derivative is used alone, polymeric micelles can be formed regardless of the pH of the aqueous medium when the amphiphilic block copolymer is added. Therefore, since biodegradable polymers are generally hydrolyzed at a pH of 10 or higher, the polymeric composition can be used in the range of from pH 1-10, preferable pH 4-8 because the polymers are biodegradable polymers.

The pharmaceutical composition containing the active ingredient, itraconazole, can form polymeric micelles regardless of the pH of the aqueous medium when the composition comprises 0.1-30.0 wt. % of itraconazole, 5.0-99.8 wt. % of a polylactic acid derivative having at least one terminal carboxyl group, and 0.1-94.9 wt. % of an amphiphilic block copolymer.

Preferably, the pharmaceutical composition of the present invention comprises 0.1-20.0 wt. % of itraconazole, 20-80 wt. % of the polylactic acid derivative having at least one terminal carboxyl group, and 5.0-79.9 wt. % of the amphiphilic block copolymer.

The amphiphilic block copolymer contained in the pharmaceutical composition of the present invention is an A-B type diblock copolymer comprising a hydrophilic A-block component (A) and a hydrophobic B-block component (B), and is nonionic. When placed in an aqueous phase, the amphiphilic block copolymer forms core-shell-typed polymeric micelles wherein the hydrophobic block (B) occupies the inner core and the hydrophilic block (A) forms the shell. A composition ratio between hydrophilic and hydrophobic block in the amphiphilic block copolymer is 2-8:8-2 (w/w), preferably 4-7:6-3 (w/w).

Examples of the hydrophilic A block, which is a water-soluble polymer, includes polyalkylene glycol, polyethylene glycol, polyethylene-co-propylene glycol, polyvinyl pyrrolidone, polyvinyl alcohol, polyacrylamide, monomethoxy polyalkylene glycol, and monoacetoxypolyethylene glycol. Preferably, the hydrophilic block is monomethoxy polyalkylene glycol. The hydrophilic block preferably has a number-average molecular weight of 1,000-10,000 Daltons. More preferably, the hydrophilic block has a number-average molecular weight of 1,000-5,000 Daltons.

The water-insoluble hydrophobic B block is highly biocompatible and biodegradable, and examples include polylactides, polyglycolides, polydioxane-2-one, polycaprolactone, polylactic-co-glycolide, polylactic-co-caprolactone, polylactic-co-dioxane-2-one, poly D-lactic acid, poly L-lactic acid, and poly DL-lactic acid, preferably poly D-lactic acid and poly DL-lactic acid. The hydroxyl terminal group of the hydrophobic B block can be substituted with a fatty acid group such as butyric acid, propionic acid, acetic acid, stearic acid and palmitic acid. The hydropobic block preferably has a number-average molecular weight of 1,000-10,000 Daltons, more preferably 1,000-5,000 Daltons.

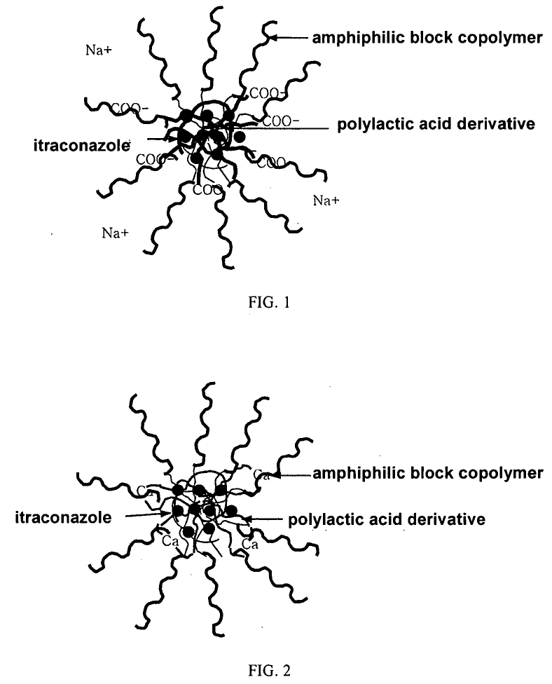

As shown in FIG. 1, the carboxyl group positioned at the terminus of the polylactic acid derivative is reacted first with the amine group of itraconazole to form a polymer-drug ionic complex and the complex is entrapped tightly into the hydrophobic core of the micelle or nano-particle which is comprised of the amphiphilic block copolymer of the present invention. And the polylactic acid derivative having at least one terminal carboxyl group is inserted between the amphiphilic block copolymers. The polymer-drug ionic complex allows itraconazole to be stable, and not be degraded in an aqueous medium. The stability of hydrophobic core composed of drug ionic complex and hydrophobic B block of amphiphilic block copolymer is increased due to interaction between drug and polylactic acid derivative. According to the present invention, itraconazole was not solubilized with the amphiphilic block copolymer only which has been generally known to solubilize water insoluble drugs, but itraconazole can be solubilized with the polylactic acid derivative only or the polylactic acid derivative and the amphiphilic block copolymer.

The particle size of the micelles in the present invention may be controlled depending on the molecular weight of the polymer used and the composition ratio between the polylactic acid derivative and the amphiphilic block copolymer. It is preferably within the range of 10-200 nm, more preferably 10-100 nm.

Furthermore, the pharmaceutical composition comprises 0.1-30.0 wt. % of itraconazole, 5.0-99.8 wt. % of a polylactic acid derivative having at least one terminal carboxyl group wherein said carboxyl group is fixed with di- or tri-valent metal ion, and 0.1-94.9 wt. % of an amphiphilic block copolymer comprised of a hydrophilic block and a hydrophobic block.

More preferably, the pharmaceutical composition comprises 0.1-20.0 wt. % of itraconazole, 20-70 wt. % of a polylactic acid derivative having at least one terminal carboxyl group wherein said carboxyl group is fixed with 0.5-4.0 equivalents of a di- or tri-valent metal ion per equivalent of carboxyl group, and 10-79.9 wt. % of an amphiphilic block copolymer.

Features of the polylactic acid derivative and amphiphilic block copolymer used are the same as described above. Particularly, the terminal carboxyl group of the polylactic acid derivative is combined with a di- or tri-valent metal ion where 0.5-4.0 equivalents of di- or tri-valent metal ion per equivalent of carboxyl group are combined. Examples of the metal ions include Ca²⁺, Mg²⁺, Ba²⁺, Mn²⁺, Ni²⁺, Cu²⁺, Zn²⁺, Cr³⁺, Fe³⁺, and Al³⁺, more preferably Ca²⁺.

The metal ion is added as its sulfate, carbonate, phosphate, or hydroxylate into the polymer composition of the polylactic acid and amphiphilic block copolymer, more preferably, as CaCl₂, MgCl₂, ZnCl₂, AlCl₃, FeCl₃, CaCO₃, MgCO₃, Ca₃(PO₄)₂, Mg₃(PO₄)₂, AlPO₄, MgSO₄, Ca(OH)₂, Mg(OH)₂, Al(OH)₃ or Zn(OH)₂.

As shown in FIG. 2, when the di- or tri-valent metal ions are substituted with monovalent metal cations at the terminal carboxyl group of the polylactic acid, the polymeric micelles or nano-particles formed have much improved stability.

0.5-4.0 equivalents of the di- or tri-valent metal ion are preferably used per equivalent of carboxyl group of the polylactic acid derivative, more preferably 0.5-2.0 equivalents are used. Equivalents of the di- or tri-valent metal ion may control the release rate of itraconazole entrapped in the polymeric micelle or nano-particle. That is, if less than 0.5 equivalents of di- or tri-valent metal ion per equivalent of carboxyl group of the polylactic acid derivative are used, the release rate of itraconazole increases because the number of di- or tri-valent metal ions which react with the terminal carboxyl group of the polylactic acid derivative are less. On the other hand, if more than 4.0 equivalents are used, the release rate of itraconazole is delayed because the number of di- or tri-valent metal ions which react with the terminal carboxyl group of the polylactic acid derivative is increased. Therefore, fewer equivalents of the metal ion are used to increase the release rate of the drug and more equivalents cause a delayed release.

Furthermore, the present invention includes a process for preparing the above pharmaceutical composition. A particular ratio of itraconazole and polylactic acid derivative or itraconazole, polylactic acid derivative and amphiphilic block copolymer is dissolved in an organic solvent. The solvent is evaporated and the mixture obtained is added to an aqueous medium to prepare polymeric micelles containing itraconazole.

The polymeric micelle or nano-particle fixed with di- or tri-valent metal ions may be prepared by adding di- or tri-valent metal ions to the above polymeric micelles, thereby fixing the terminal carboxyl group of the polylactic acid derivative.

A particular ratio of itraconazole and polylactic acid derivative or itraconazole, polylactic acid derivative and amphiphilic block copolymer is dissolved in one solvent selected from the group consisting of acetone, ethanol, methanol, ethyl acetate, acetonitrile, methylene chloride, chloroform, acetic acid, dioxane, or a mixture thereof. The solvent is evaporated to obtain a homogeneous mixture containing itraconazole. The mixture is added to an aqueous medium having a pH of 4-8 and polymeric micelles containing itraconazole form at a temperature of 0-60° C. The above itraconazole-containing polymeric micelles can then be lyophilized to produce a solid form of the polymeric micelle.

Furthermore, an aqueous solution containing 0.001 to 2M of di- or tri-valent metal ion is added to the mixed polymeric micelle aqueous solution. The mixture is slowly stirred for 0.1-1 hrs at room temperature and then lyophilized to produce the solid form of polymeric micelles or nano-particles fixed with di- or tri-valent metal ions. The solid form of the above composition may be reconstituted for injection with an aqueous medium such as water, saline or 5% dextrose solution.

The composition of the present invention may additionally include pharmaceutically acceptable excipients such as stabilizers, coloring agents, preserving agents and the like.

The particle size of the present invention in an aqueous medium is preferably within the range of 10-200 nm, more preferably 10-100 nm since a fine particle size is desirable for oral and intravenous injection preparations.

The itraconazole of the present invention is entrapped in the core of the polymeric micelles or nano-particles and then administered orally or parenterally to human or animals. Especially, when the polymeric micelles or nano-particles fixed with di- or tri-valent metal ions are administered into the body, they are retained longer in the blood stream, increasing the pharmacological effect of itraconazole, and remarkably reducing the dose administered.

The pharmaceutical composition of the present invention may be used in the treatment of mycosis such as blastomycosis, histoplasmosis, aspergillosis, onychomycosis, and the like. The amount of itraconazole administered will be determined in light of the relevant circumstances, including the patient's age, weight, and sex, the condition to be treated, the severity of the patient's symptoms, and the like. Suitable doses of itraconazole are from 50-100 mg per day to 800-1000 mg per day.

The pharmaceutical composition of the present invention can be parenterally administered through blood vessels, muscle, hypodermis, abdomen, nose, rectum, eye, lung or orally administered in the form of tablets, capsules or solutions.

The amphiphilic block copolymer and polylactic acid derivative used in the present invention are prepared by the process disclosed in U.S. patent application Ser. No. 10/492,091 and 10/493,043, which are fully incorporated herein by reference.

The following examples will enable those skilled in the art to more clearly understand how to practice the present invention. It should be understood that even though the invention has been described in conjunction with the preferred specific embodiments thereof, the following is not intended to limit the scope of the present invention. Other aspects of the invention will be apparent to those skilled in the art to which the invention pertains.

### EXAMPLES 1 and 2

### Preparation of Micelle With Itraconazole and a Polylactic Acid Derivative

Itraconazole and a polylactic acid derivative in the amount described in Table 1 were completely dissolved in 2 mL of methylene chloride which was then evaporated. The residue was dissolved in distilled water and sodium hydrogen carbonate was added to give a pH of 4.0-7.0. The mixture was filtered through a filter having a pore size of 200 nm and lyophilized to produce the powder form of the polymeric micelle composition containing itraconazole.

### EXAMPLES 3-8

### Preparation of Micelle With Itraconazole, a Polylactic Acid Derivative, and the Amphiphilic Block Copolymer

Itraconazole, a polylactic acid derivative, and the amphiphilic block copolymer in the amount described in Table 1 were completely dissolved in 2 mL of methylene chloride which was then evaporated. The residue was dissolved in distilled water and sodium hydrogen carbonate was added to give a pH of 4.0-7.0. The mixture was filtered through a filter having a pore size of 200 nm and lyophilized to produce a powder form of the polymeric micelle composition containing itraconazole.

### Comparative Example 1

Itraconazole and the amphiphilic block copolymer in the amount described in Table 1 were completely dissolved in 2 mL of methylene chloride which was then evaporated. The residue was dissolved in distilled water and sodium hydrogen carbonate was added to give a pH of 4.0-7.0. The mixture was filtered through a filter having a pore size of 200 nm and lyophilized to produce a powder form of the polymeric micelle composition containing itraconazole.

TABLE 1

| | Composition(%) | | |
|---|---|---|---|
| | | Derivative | Amphiphilic block |
| Example | Itraconazole | of polylactic acid¹⁾ | copolymer²⁾ |
| Ex. 1 | 15.0 | 85.0 | — |
| Ex. 2 | 5.0 | 95.0 | — |
| Ex. 3 | 15.0 | 65.5 | 19.5 |
| Ex. 4 | 15.0 | 47.5 | 37.5 |
| Ex. 5 | 15.0 | 42.5 | 42.5 |
| Ex. 6 | 15.0 | 25.0 | 60.0 |
| Ex. 7 | 5.0 | 47.5 | 47.5 |
| Ex. 8 | 8.0 | 50.0 | 42.0 |
| Com. Ex. 1 | 15.0 | — | 85.0 |

¹⁾D,L-PLA-COONa, number-average molecular weight(Mn) 1,150 Daltons
²⁾Monomethoxy polyethylene glycol-polylactide, Mn 2,000-1,766 Daltons

### EXAMPLES 9-11

### Preparation of Nano-Paricle Fixed With Metal Ions

Step 1: Itraconazole, a polylactic acid derivative, and the amphiphilic block copolymer in the amount described in Table 2 were completely dissolved in 2 mL of methylene chloride which was then evaporated. The residue was dissolved in distilled water to produce a micelle solution.

Step 2: 0.1 M anhydrous calcium chloride aqueous solution was added to the micelle solution prepared in Step 1. The mixture was stirred for 20 min at room temperature and sodium hydrogen carbonate was added to give a pH of 4.0-7.0. The mixture was filtered through a filter having a pore size of 200 nm and the freeze-dried to produce the powder form of the polymeric nano-particle composition containing itraconazole.

TABLE 2

| | Used amount (wt %) | | | |
|---|---|---|---|---|
| | | Derivative of | Amphiphilic | |
| | | polylactic | block | |
| Ex. | Itraconazole | acid¹⁾ | copolymer²⁾ | Metal ion salt³⁾ |
| Ex. 9 | 4.9 | 46.4 | 46.4 | 2.3 |
| Ex. 10 | 14.9 | 24.7 | 59.3 | 1.1 |
| Ex. 11 | 14.7 | 46.4 | 36.7 | 2.2 |

¹⁾D,L-PLA-COONa, number-average molecular weight(Mn) 1,150 Daltons
²⁾Monomethoxy polyethylene glycol-polylactide, Mn 2.000-1,766 Daltons
³⁾Calcium chloride

### Comparative Example 2

Itraconazole (100 mg) and hydroxypropyl-β-cyclodextrin (4 g) were dissolved in a solution of ethanol and methylene chloride and the solvent was evaporated in order to give a uniform mixture. The mixture was then dissolved in distilled water and propylene glycol (250 mg) and hydrochloric acid (38 μl) were added thereto. The mixture solution was then filtered through a filter having a pore size of 200 nm and freeze-dried to produce an itraconazole composition solubilized in cyclodextrin.

### Experimental Example 1

### Determination of Loading Efficiency of Itraconoazole and Particle Size

The amount of itraconazole contained in polymeric micelles or nano-particles prepared in Examples 1-11 and Comparative Example 1 was determined according to HPLC conditions described in Table 3 and the amount was converted to weight. Furthermore, particle size was determined by employing dynamic light scattering (DLS). Loading efficiency was calculated by the following equation. The results are summarized in Table 4.

Presumed loading amount (%)=[amount of itraconazole used/(amount of itraconazole used+amount of polymer used+amount of metal ion used)]×100 Real loading amount (%)=(weight of itraconazole/weight of a sample)×100 Loading efficiency (%)=(weight of entrapped itraconazole/amount of itraconazole used)×100

TABLE 3

| Category | Condition |
|---|---|
| Eluent | 54% acetonitrile/46% water/0.05% diethyl amine |
| Column | C18, inner diameter 4 mm, length 25 cm (Vydak, USA) |
| Wave length | 258 nm |
| Rate | 0.8 mL/min |
| Temperature | Room Temp. |
| Volume | 80 μl |
| Retention Time | 18 min |

TABLE 4

| | Itraconozole | | | |
|---|---|---|---|---|
| | presumed | Itraconozole | Itraconozole | |
| | loading | real loading | loading efficiency | Particle |
| Category | amount (%) | amount (%) | (%) | size (nm) |
| Ex. 1 | 15.0 | 12.5 | 83.3 | 100 |
| Ex. 2 | 5.0 | 4.6 | 92.0 | 80 |
| Ex. 3 | 15.0 | 13.6 | 90.1 | 100 |
| Ex. 4 | 15.0 | 14.5 | 96.7 | 36 |
| Ex. 5 | 15.0 | 14.2 | 94.7 | 34 |
| Ex. 6 | 15.0 | 14.5 | 96.7 | 27 |
| Ex. 7 | 5.0 | 4.7 | 94.0 | 25 |
| Ex. 8 | 8.0 | 7.4 | 92.5 | 28 |
| Ex. 9 | 4.9 | 4.5 | 91.8 | 20 |
| Ex. 10 | 15.0 | 14.5 | 96.7 | 32 |
| Ex. 11 | 14.7 | 14.1 | 95.9 | 28 |
| Com. Ex. 1 | 15.0 | 0.5 | 3.3 | — |

As shown in Table 4, it is noted that the loading efficiency of the present invention is 80% or above compared to that of the composition solubilized only with the amphiphilic block copolymer (Comparative Example 1). The solubility of itraconazole in Examples 1-11 was more than 10 mg/mL and the particle size in an aqueous medium is less than 100 nm which is thus suitable for intravenous injection preparations.

### Experimental Example 2

### Stability Test

The compositions from Examples 2, 7 and 9 were filtered through a filter having a pore size of 200 nm and the filtrate was diluted with distilled water until the concentration of itraconazole was 3.3 mg/mL. Then, the concentration of itraconazole was measured by HPLC while incubating at 25° C., with the conditions shown in Table 3, at the given time intervals. The results are summarized in Table 5.

Residual itraconazole (%)=(content of itraconazole after storage/initial content of itraconazole)×100

TABLE 5

| | | Residual Itraconazole (%) | | |
|---|---|---|---|---|
| Category | | Ex. 2 | Ex. 7 | Ex. 9 |
| Time(hr) | 0 | 100.0 | 100.0 | 100.0 |
| | 2 | 98.0 | 99.2 | 99.6 |
| | 24 | 93.0 | 95.2 | 97.6 |

As shown in Table 5, it is noted that the amount of itraconazole in the aqueous medium of the present invention was 90% or above for 24 hrs and the micelle composition using the polylactic acid derivative and the amphiphilic block copolymer having a carboxyl terminal group (Examples 7 and 9) is more stable than the micelle composition using the polylactic acid derivative alone (Example 2). The polymeric nano-particle composition using methoxypolyethylene glycol-polylactide and calcium ions as an amphiphilic block copolymer (Example 9) exhibits far better stability.

### Experimental Example 3

### Test for Bioavailability Using Intravenous Administration

Male Sprague-Dawley rats having weights of 230-250 g were purchased and monitored for 2 weeks before being used in the experiment. The composition prepared in Example 9 or Comparative Example 2 was used for the test.

The male rats were cannulated in the vena femoralis and aorta femoralis and the composition was injected in the vena femoralis at a dose of 10 mg/kg of itraconazole over 15 seconds. After injection, 0.3 mL of whole blood was taken from the aorta femoralis at 1, 5, 15, 30, 60, 120, 180, and 300 minutes and then, centrifuged to obtain clear supernatant plasma.

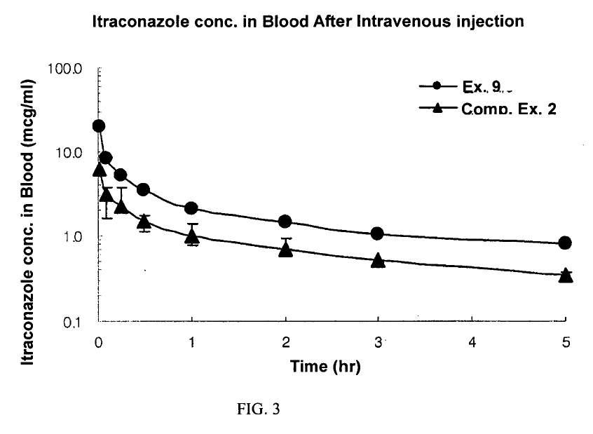

To analyze the plasma concentration of drug, 100 μl of the plasma was introduced into a covered glass tube and an acetonitrile solution containing 0.1 μl ketoconazole as an internal standard was added thereto. 50 μl of 0.1 M carbonate buffer solution (pH 10) and 7 mL of a heptane/isoamylalcohol (90/10) mixture solution were added to the above solution. The mixture was vigorously shaken for 10 seconds then lightly shaken for 10 minutes and then, centrifuged at 1,200 rpm for 10 minutes. The clear upper layer was removed and the solvent was completely evaporated at 40° C. under nitrogen flow. Thereto was added 125 μl of a HPLC mobile phase and the mixture was then subjected to HPLC with the conditions described in Table 3. The results are illustrated in FIG. 3.

As shown in FIG. 3, when the composition of Example 9 was intravenously administered, the plasma concentration of drug always exceeded the effective blood concentration. It is also noted that the composition of Example 9 exhibited higher plasma concentrations of drug than the composition of Comparative Example 2 which solubilized itraconazole by using hydroxypropyl-β-cyclodextrin.

### Experimental Example 4

### Test for Bioavailability Using Oral Administration

Male Sprague-Dawley rats having weights of 230-250 g were purchased and monitored for 2 weeks before being used in the experiment. The composition prepared in Example 2 or Comparative Example 2 was used for the test.

The compositions were administered orally at a dose of 10 mg/kg of itraconazole to, two groups of rats that had fasted for 24 hours.

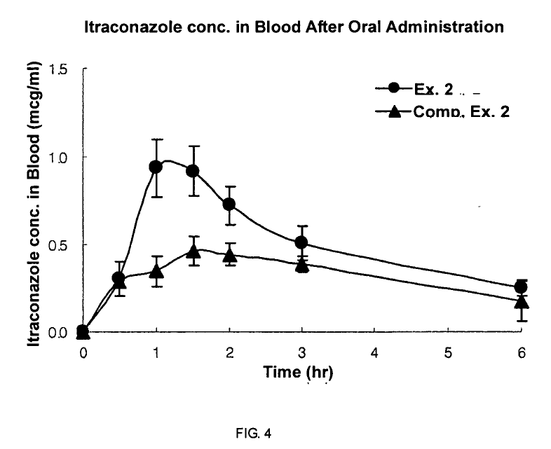

After administration, blood was taken from the tail vein at 30, 60, 120, 180, and 300 minutes and then, the bioavailability was analyzed with the same procedure conducted in Experimental Example 3. The results are illustrated in FIG. 4.

As shown in FIG. 4, when the composition of Example 2 was orally administered, the plasma concentration of drug reached the effective blood concentration after 30 minutes. It is also noted that the composition of Example 2 exhibited better bioavailability than the composition of Comparative Example 2 wherein itraconazole was solubilized by using hydroxypropyl-β-cyclodextrin.

As described above, the pharmaceutical composition containing as an active ingredient, itraconazole, of the present invention forms polymeric micelles or nano-particles in an aqueous medium which improves solubility and is suitable for entrapping a large amount. Furthermore, the composition can exist in the form of fine particles in an aqueous medium and be suitable for intravenous injection due to high stability. It also has longer bloodstream retention time due to improved stability which results in an increased pharmacological effect and allows for remarkably reduced doses of administration to be given.

It is to be understood that the above-described embodiments are only illustrative of application of the principles of the present invention. Numerous modifications and alternative embodiments can be derived without departing from the spirit and scope of the present invention, and the appended claims are intended to cover such modifications and arrangements. Thus, while the present invention has been shown in the drawings and is fully described above with particularity and detail in connection with what is presently deemed to be the most practical and preferred embodiment(s) of the present invention, it will be apparent to those of ordinary skill in the art that numerous modifications can be made without departing from the principles and concepts of the present invention as set forth in the claims.

1. A pharmaceutical composition containing itraconazole as an active ingredient comprising:
0.1-30.0 wt. % of itraconazole and
70.0-99.9 wt. % of a polylactic acid derivative having at least one terminal carboxyl group,
wherein said composition forms polymeric micelles or nano-particles in an aqueous medium which improves solubility and stability of the itraconazole.

2. A pharmaceutical composition containing itraconazole as an active ingredient comprising:
0.1-30.0 wt. % of itraconazole,
5.0-99.8 wt. % of a polylactic acid derivative having at least one terminal carboxyl group, and
0.1-94.9 wt. % of an amphiphilic block copolymer comprised of a hydrophilic block and a hydrophobic block,
wherein said composition forms polymeric micelles or nano-particles in an aqueous medium which improves solubility and stability of the itraconazole.

3. The pharmaceutical composition of claim 1, wherein said composition forms micelles in an aqueous medium.

4. The pharmaceutical composition of claim 2, wherein said composition forms micelles in an aqueous medium.

5. The pharmaceutical composition of claim 1, wherein said polylactic acid derivative has a number-average molecular weight of 500-2,500 Daltons.

6. The pharmaceutical composition of claim 2, wherein said polylactic acid derivative has a number-average molecular weight of 500-2,500 Daltons.

7. The pharmaceutical composition of claim 1, wherein said polylactic acid derivative is selected from the group consisting of D,L-polylactic acid, D-polylactic acid, polymandelic acid, a copolymer of D,L-lactic acid and glycolic acid, a copolymer of D,L-lactic acid and mandelic acid, a copolymer of D,L-lactic acid and caprolactone, a copolymer of D,L-lactic acid and 1,4-dioxane-2-one, and acyl-D,L-lactic acid substituted with a C₈-C₁₄ acyl, wherein at least one end of the polylactic acid derivative is covalently bonded to at least one carboxylic acid or alkali metal ion thereof.

8. The pharmaceutical composition of claim 2, wherein said polylactic acid derivative is selected from the group consisting of D,L-polylactic acid, D-polylactic acid, polymandelic acid, a copolymer of D,L-lactic acid and glycolic acid, a copolymer of D,L-lactic acid and mandelic acid, a copolymer of D,L-lactic acid and caprolactone, a copolymer of D,L-lactic acid and 1,4-dioxane-2-one, and acyl-D,L-lactic acid substituted with a C₈-C₁₄ acyl, wherein at least one end of the polylactic acid derivative is covalently bonded to at least one carboxylic acid or alkali metal ion thereof.

9. The pharmaceutical composition of claim 2, wherein said hydrophilic block is a member selected from the group consisting of polyalkylene glycol, polyethylene glycol, polyethylene-co-propylene glycol, polyvinyl pyrrolidone, polyvinyl alcohol, polyacrylamide, monomethoxy polyalkylene glycol and monoacetoxypolyethylene glycol.

10. The pharmaceutical composition of claim 2, wherein said hydrophobic block is a member selected from the group consisting of polylactides, polyglycolides, polydioxane-2-one, polycaprolactone, polylactic-co-glycolide, polylactic-co-caprolactone, polylactic-co-dioxane-2-one, poly D-lactic acid, poly L-lactic acid and poly DL-lactic acid.

11. The pharmaceutical composition of claim 2, wherein said hydrophilic block and hydrophobic block have number-average molecular weights of 1,000-10,000 Daltons each.

12. The pharmaceutical composition of claim 2, wherein the ratio of said hydrophilic block to said hydrophobic block of said amphiphilic block copolymer is 2-8 to 8-2 (w/w).

13. The pharmaceutical composition of claim 2, comprising 0.1-20.0 wt. % of said itraconazole, 20-80 wt. % of said polylactic acid derivative, and 5.0-79.9 wt. % of said amphiphilic block copolymer.

14. A pharmaceutical composition containing itraconazole as an active ingredient comprising:
0.1-30.0 wt. % of itraconazole,
5.0-99.8 wt. % of a polylactic acid derivative having at least one terminal carboxyl group
wherein said carboxyl group is fixed with a di- or tri-valent metal ion, and
0.1-94.9wt. % of an amphiphilic block copolymer comprised of a hydrophilic block and a hydrophobic block,
wherein said composition forms polymeric micelles or nano-particles in an aqueous medium which improves solubility and stability of the itraconazole.

15. The pharmaceutical composition of claim 14, wherein said composition forms micelles or nano-particles in an aqueous medium.

16. The pharmaceutical composition of claim 14, wherein said hydrophilic block is a member selected from the group consisting of polyalkylene glycol, polyethylene glycol, polyethylene-co-propylene glycol, polyvinyl pyrrolidone, polyvinyl alcohol, polyacrylamide, monomethoxy polyalkylene glycol and monoacetoxypolyethylene glycol.

17. The pharmaceutical composition of claim 14, wherein said hydrophobic block is selected from the group consisting of polylactides, polyglycolides, polydioxane-2-one, polycaprolactone, polylactic-co-glycolide, polylactic-co-caprolactone, polylactic-co-dioxane-2-one, poly D-lactic acid, poly L-lactic acid, and poly DL-lactic acid.

18. The pharmaceutical composition of claim 14, wherein said hydrophilic block and hydrophobic block have number-average molecular weights of 1,000-10,000 Daltons each.

19. The pharmaceutical composition of claim 14, wherein the ratio of said hydrophilic block to said hydrophobic block of said amphiphilic block copolymer is 2-8 to 8-2 (w/w).

20. The pharmaceutical composition of claim 14, wherein said di- or tri-valent metal ion is selected from the group consisting of Ca²⁺, Mg²⁺, Ba²⁺, Mn²⁺, Ni²⁺, Cu²⁺, Zn²⁺, Cr³⁺, Fe³⁺, and Al³⁺.

21. The pharmaceutical composition of claim 14, wherein said metal ion is used in the range of 0.5-4.0 equivalents per equivalent of the terminal carboxyl group of said polylactic acid derivative.

22. The pharmaceutical composition of claim 14, comprising:
0.1 -20.0 wt. % of itraconazole,
20-70 wt. % of a polylactic acid derivative having at least one terminal carboxyl group
wherein said carboxyl group is fixed with 0.5-4.0 equivalents of di- or tri-valent metal ion per equivalent of carboxyl group, and
10-79.9 wt. % of said amphiphilic block copolymer of said hydrophilic block and said hydrophobic block.

23. A method for effectively administering itraconazole to a warm-blooded animal in need of said treatment, comprising the step of making a pharmaceutical composition containing itraconazole as an active ingredient comprising:
0.1-30.0 wt. % of itraconazole and
70.0-99.9 wt. % of a polylactic acid derivative having at least one terminal carboxyl group,
wherein said composition forms polymeric micelles or nano-particles in an aqueous medium which improves solubility and stability of the itraconazole.

24. A method for effectively administering itraconazole to a warm-blooded animal in need of said treatment, comprising the step of making a pharmaceutical composition containing itraconazole as an active ingredient comprising:
0.1-30.0 wt. % of itraconazole,
5.0-99.8 wt. % of a polylactic acid derivative having at least one terminal carboxyl group, and
0.1-94.9 wt. % of an amphiphilic block copolymer comprised of a hydrophilic block and a hydrophobic block,
wherein said composition forms polymeric micelles or nano-particles in an aqueous medium which improves solubility and stability of the itraconazole.

25. A method for effectively administering itraconazole to a warm-blooded animal in need of said treatment, comprising the step of making a pharmaceutical composition containing itraconazole as an active ingredient comprising:
0.1-30.0 wt. % of itraconazole,
5.0-99.8 wt. % of a polylactic acid derivative having at least one terminal carboxyl group
wherein said carboxyl group is fixed with a di- or tri-valent metal ion, and
0.1-94.9 wt. % of an amphiphilic block copolymer comprised of a hydrophilic block and a hydrophobic block,
wherein said composition forms polymeric micelles or nano-particles in an aqueous medium which improves solubility and stability of the itraconazole.
